SEQUENCE LISTING (5 sequences)

SEQ ID NO: 1 - protein, 7 aa, Artificial sequence
  Leptin receptor affinity peptide
HGGVRLY

SEQ ID NO: 2 - DNA, 21 bp, Artificial sequence
  Leptin receptor affinity peptide
atacaaacgaaccccaccatg

SEQ ID NO: 3 - protein, 7 aa, Artificial sequence
  CBD
TKKTLRT

SEQ ID NO: 4 - protein, 5 aa, Artificial sequence
  linker
GGGGS

SEQ ID NO: 5 - protein, 4 aa, Artificial sequence
  linker labeled fluorescence
GGGK

(19) United States
(12) Patent Application Publication
(10) Pub. No.: US 2024/0307589 A1
(43) Pub. Date: Sep. 19, 2024

(54) LEPTIN RECEPTOR AFFINITY PEPTIDE AND USE THEREOF

(71) Applicant: Dubu Biosciences, Jiangsu (CN)

(72) Inventors: Jianwu Dai, Jiangsu (CN); Yan Zhuang, Jiangsu (CN)

(21) Appl. No.: 18/571,648

(22) PCT Filed: May 18, 2022
(86) PCT No.: PCT/CN2022/093475

§ 371 Date: Dec. 18, 2023

(30) Foreign Application Priority Data
Jun. 18, 2021 (CN) 202110675456.8

(51) Int. Cl. A61L 27/22; A61L 27/24; C07K 7/06

(52) U.S. Cl. CPC A61L 27/227; A61L 27/24; C07K 7/06

(57) ABSTRACT

Disclosed in the present application are a leptin receptor affinity peptide and the use thereof. The sequence of the leptin receptor affinity peptide is as shown in SEQ ID NO. 1. Further disclosed in the present application is an affinity peptide with a collagen binding capacity, which affinity peptide comprises the leptin receptor affinity peptide and a short peptide with a collagen specific binding capacity, wherein the short peptide with the collagen specific binding capacity binds to the N-terminus of the leptin receptor affinity peptide. The affinity peptide of the present application has a high affinity with a leptin receptor and can specifically bind to MSCs. When the affinity peptide is connected to a biological material, the affinity peptide connected to the short peptide with the collagen specific binding capacity can specifically bind to a collagen scaffold, so that the recruitment of the biological material to MSCs expressing the leptin receptor on the surface is increased, a better tissue injury repair effect is obtained, and an experimental basis is provided for the research on MSCs in tissue engineering repair and targeted therapy.

### TECHNICAL FIELD

The present application belongs to the field of biomedical technology and, specifically, relates to a leptin receptor affinity peptide and, in particular, relates to a leptin receptor affinity peptide that can specifically bind to mesenchymal stem cells (MSCs) and a screening method thereof, and is applied to tissue engineering repair.

### BACKGROUND

Mesenchymal stem cells (MSCs), which are mainly derived from mesoderm, are a type of adult stem cells with a high self-renewal ability and differentiation potential, and are important members of a stem cell family. The MSCs are initially found in bone marrow, and subsequent studies have found that the MSCs are mainly present in systemic connective tissues and interstitium of organs. At present, the MSCs can be separated and prepared from tissues such as bone marrow, placenta, umbilical cord and fat. Based on the multi-differentiation potential of the MSCs, the MSCs can be directed to differentiate into cells of multiple tissues such as bone, cartilage, fat and myocardium in particular induction environments in vivo and in vitro. In addition, the MSCs have a feature of low immunogenicity so that the MSCs can avoid an immune rejection in a transplantation application. These features of the MSCs provide a new therapeutic idea and protocol for clinical treatment and can be used as most suitable seed cells in tissue engineering repair for the treatment of bone and muscle degenerative diseases, cerebrovascular diseases and autoimmune diseases.

In a development process of tissue engineering, an application of a scaffold material to adsorb the MSCs for tissue injury repair and regeneration has been widely acknowledged. However, neither MSCs that are simply added dropwise to the material exogenously nor MSCs in a circulation in vivo can stay on the scaffold material for a long time due to the fluidity of a body fluid. Therefore, the MSCs cannot perform effects of differentiation and repair. Therefore, if a surface of the scaffold material can be modified and ligated to a protein or polypeptide with a strong binding strength to the MSCs so that an adhesion rate of the MSCs on the surface of the material is improved, a regeneration ability of MSCs on the scaffold material must be better performed, and a repair effect of the scaffold material is improved. In a recent study, researchers have found that in the MSCs, a small portion of cells with a good proliferation and differentiation ability express a leptin receptor on a surface, and if an affinity peptide with a strong binding strength to the leptin receptor can be found, the affinity peptide can be modified to the surface of the scaffold material so that the affinity peptide functions as a bridge to specifically bind to this portion of MSCs with the good proliferation and differentiation ability.

How to find a leptin receptor affinity peptide, a phage display technique provides a mature solution. Numerous studies have proved that the phage display technique plays an important role in studying polypeptides or proteins in property, mutual recognition and interaction. An exogenous DNA fragment of the polypeptide is encoded, fused with a coding gene of a protein on a surface of a phage and presented in the form of fusion protein on the surface of the phage. The displayed polypeptide or protein can maintain a relative spatial structure and biological activity, and is displayed on the surface of the phage. A phage display random peptide library technique is developed on the basis of the phage display technique. When a large number of phages with different exogenous genes are introduced, a phage display library that can display a large number of different exogenous peptides is formed. An advantage of a phage display peptide library is that the phage display peptide library provides a direct physical link between a protein and genetic information so that clones of required functions can be effectively screened repeatedly and amplified, gene sequences corresponding to these specifically bound exogenous peptides are obtained and biological functions of these polypeptides can be studied at a later stage. In a process of library screening, particular phage clones are constantly enriched due to a specific affinity for ligands of the particular phage clones so that relatively rare clones that can bind to ligands can be quickly and efficiently screened out from a large library.

Therefore, how to obtain a polypeptide that has a high affinity for the leptin receptor and specifically binds to the MSCs and apply the polypeptide to the tissue engineering repair has become a direction for researchers in the industry to endeavor for a long time.

### SUMMARY

The present application provides a leptin receptor affinity peptide that can specifically bind to mesenchymal stem cells (MSCs) and use thereof.

In a first aspect, the present application provides a leptin receptor affinity peptide, wherein a sequence of the leptin receptor affinity peptide is as shown in SEQ ID No. 1.

Further, the leptin receptor affinity peptide has a sequence that is identical to more than 95% of a full-length sequence of SEQ ID No. 1.

Further, the leptin receptor affinity peptide has a high affinity for a leptin receptor and can specifically bind to mesenchymal stem cells.

In a second aspect, the present application provides a method for screening a leptin receptor affinity peptide. In the method, a phage display technique is used with a leptin receptor recombinant protein for affinity screening, and finally, a polypeptide that has a high affinity for a leptin receptor is screened out in a random heptapeptide library.

In a third aspect, the present application provides an affinity peptide with a collagen binding ability. The affinity peptide includes the leptin receptor affinity peptide and a short peptide with a collagen-specific binding ability, wherein the short peptide with the collagen-specific binding ability is ligated and bound to an N-terminus of the leptin receptor affinity peptide through a linker, a sequence of the leptin receptor affinity peptide is as shown in SEQ ID No. 1, the short peptide with the collagen-specific binding ability is a collagen binding domain (CBD) whose sequence is as shown in SEQ ID No. 3, and a sequence of the linker is as shown in SEQ ID No. 4 or SEQ ID No. 5.

In a fourth aspect, the present application provides a functional material. The functional material includes a collagen material and the affinity peptide with the collagen binding ability, wherein the affinity peptide with the collagen binding ability binds to a surface and/or an interior of the collagen material.

Further, the affinity peptide with the collagen binding ability specifically binds to the collagen material through a short peptide with a collagen-specific binding ability.

In a fifth aspect, the present application provides use of the functional material according to the fourth aspect to preparation of a product with a tissue injury repair function.

In a sixth aspect, the present application provides a product with a tissue injury repair function. The product includes the functional material according to the fourth aspect.

Further, the product has at least a function of being capable of continuously adsorbing mesenchymal stem cells.

Compared with the prior art, the present application has the beneficial effects described below.

The leptin receptor affinity peptide provided in the present application has the high affinity for the leptin receptor and can specifically bind to the MSCs. The leptin receptor affinity peptide can be ligated to a biological material so that the affinity polypeptide ligated to the short peptide with the collagen-specific binding ability can specifically bind to a collagen scaffold, thereby improving the recruitment of the biological material to MSCs expressing the leptin receptor on a surface, obtaining a better tissue injury repair effect and providing an experimental basis for studies on the MSCs in tissue engineering repair and targeted therapy study.

### BRIEF DESCRIPTION OF DRAWINGS

To illustrate the technical solutions in the examples of the present application or the technical solutions in the existing art more clearly, drawings used in the description of the examples or the existing art will be briefly described below. Apparently, the drawings described below illustrate only part of the examples recorded in the present application, and those of ordinary skill in the art may obtain other drawings based on the drawings described below without any creative work.

FIG. 1 is a schematic diagram illustrating the binding recovery of an HY7 phage monoclone and an M13KE negative control phage to a leptin receptor recombinant protein in Example 1 of the present application.

FIG. 2A is a schematic diagram illustrating the binding recovery of an HY7 phage monoclone and an M13KE negative control phage to human MSCs in Example 2 of the present application, and FIG. 2B is a schematic diagram illustrating the binding recovery of an HY7 phage monoclone and an M13KE negative control phage to HS-5 cells in Example 2 of the present application.

FIG. 3 is a schematic diagram illustrating the binding of FITC-HY7 and FITC-M7 to human MSCs shot by a confocal fluorescence microscope using a polypeptide-unadded group as a control in Example 2 of the present application.

FIG. 4 is a schematic diagram illustrating the binding of FITC-HY7 and FITC-M7 to rat MSCs detected by flow cytometry in Example 2 of the present application.

FIG. 5 is a schematic diagram illustrating the binding of CBD-HY7, sCBD-HY7 and CBD-sHY7 to collagen sponges shot by a laser confocal fluorescence microscope in Example 3 of the present application.

FIGS. 6A to 6F are diagrams illustrating proportions that MSCs account for in cells digested from collagen sponges detected by flow cytometry in Example 3 of the present application, respectively.

FIG. 7 is a diagram illustrating results of Masson's staining of lung tissues and collagen sponge regions in Example 3 of the present application.

### DETAILED DESCRIPTION

The present application mainly provides an amino acid sequence, screening method and use of a leptin receptor affinity peptide that can specifically bind to MSCs, and relates to the fields of cells and polypeptide biotechnology. In the present application, a phage display technique is used with a leptin receptor recombinant protein for affinity screening, a polypeptide that has a high affinity for a leptin receptor is finally screened out in a random heptapeptide library, and that the leptin receptor affinity peptide can specifically bind to the MSCs is verified through multiple detection methods. The technical solutions and implementation processes and principles of the technical solutions will be further explained and illustrated below.

One aspect of an embodiment of the present application provides a leptin receptor affinity peptide (which may be named “HY7 peptide”). The leptin receptor affinity peptide has an amino acid sequence as shown in SEQ ID No. 1, specifically, SEQ ID No. 1: HGGVRLY.

Further, the leptin receptor affinity peptide has a sequence that is identical to more than 95% of a full-length sequence of SEQ ID No. 1.

Further, a DNA sequence of the leptin receptor affinity peptide is SEQ ID NO. 2: ATACAAACGAACCCCACCATG.

Further, the leptin receptor affinity peptide has a high affinity for a leptin receptor and can specifically bind to MSCs.

Further, the leptin receptor affinity peptide is obtained by using a phage display technique with a leptin receptor recombinant protein for affinity screening followed by screening in a random heptapeptide library.

Another aspect of an embodiment of the present application further provides a screening method for a leptin receptor affinity peptide. In the method, a surface of a phage is used for displaying a random heptapeptide library, an amino acid sequence of a heptapeptide that can specifically bind to a leptin receptor is screened out, and the specific binding of the heptapeptide to MSCs is identified.

In some embodiments, in the screening method, a phage surface display technique is used with a leptin receptor recombinant protein for affinity screening, a polypeptide that has a high affinity for the leptin receptor is finally screened out in the random heptapeptide library, and that the leptin receptor affinity peptide can specifically bind to the MSCs is verified through multiple detection methods.

In some more preferred embodiments, in the screening method, the surface of the phage is used for displaying the random heptapeptide library, and the leptin receptor recombinant protein is used as a target for five rounds of biological screening. 15 phage monoclones are randomly selected in each of the third, fourth and fifth rounds, rapid purification of a sequencing module is performed on the phage monoclones after amplification to produce a sufficiently pure template to be sent to a biological company for gene sequencing, corresponding displayed peptide sequences of the randomly selected phage monoclones are analyzed, growth trends of proportions of peptide sequences that occur repeatedly are analyzed, several phage monoclones with a relatively high proportion of occurrence are selected out, and binding abilities of the phage monoclones to the leptin receptor recombinant protein are detected. Binding abilities of these phage monoclones and synthetic corresponding polypeptides to the MSCs are detected, and a heptapeptide that has a high affinity for the leptin receptor and specifically binds to the MSCs is obtained. The heptapeptide, which has a sequence as shown in SEQ ID NO. 1, is named “HY7 peptide”

Another aspect of an embodiment of the present application further provides an affinity peptide with a collagen binding ability. The affinity peptide includes the leptin receptor affinity peptide and a short peptide with a collagen-specific binding ability, wherein the short peptide with the collagen-specific binding ability is ligated and bound to an N-terminus of the leptin receptor affinity peptide through a linker.

The leptin receptor affinity peptide has an amino acid sequence as shown in SEQ ID No. 1, specifically, SEQ ID No. 1: HGGVRLY.

Further, the short peptide with the collage-specific binding ability is a CBD whose sequence is SEQ ID No. 3: TKKTLRT.

Further, in the present application, the short peptide CBD with the collagen-specific binding ability is introduced while the affinity polypeptide is synthesized. The short peptide CBD is synthesized on the N-terminus of the leptin receptor affinity peptide and ligated to the leptin receptor affinity peptide through the linker. When the linker is not labeled with fluorescence, a sequence is SEQ ID No. 4: GGGGS. When the linker is labeled with fluorescence, the sequence is SEQ ID No. 5: GGG-K-FITC.

Another aspect of an embodiment of the present application further provides a functional material. The functional material includes a collagen material and the affinity peptide with the collagen binding ability, wherein the affinity peptide with the collagen binding ability binds to a surface and/or an interior of the collagen material.

Further, the affinity peptide with the collagen binding ability specifically binds to the collagen material through a short peptide with a collagen-specific binding ability.

Another aspect of the present application provides a use that a collagen sponge scaffold implanted into a body injury site is modified by an HY7 peptide and a modification method thereof. Obtaining the leptin receptor affinity peptide makes it possible to specifically adsorb and recruit MSCs. However, how to recruit the MSCs to a biological material through the HY7 peptide remains a problem. A simple adsorption method may cause the diffusion of a polypeptide due to the infiltration and flow of a body fluid, resulting in a reduced effective concentration of an affinity polypeptide on a part of the biological material and affecting an effect of adsorbing and recruiting the MSCs. For the technical problem, in the present application, a short peptide CBD with a collagen-specific binding ability is introduced while the leptin receptor affinity peptide is synthesized. The short peptide CBD is synthesized on an N-terminus of the affinity polypeptide and ligated to the affinity polypeptide through a linker. The affinity polypeptide ligated to the CBD can specifically bind to a collagen scaffold, thereby increasing a distribution concentration of the HY7 peptide on the collagen scaffold, enhancing a binding ability of the collagen scaffold in the body to the MSCs and obtaining a better tissue injury repair effect.

Further, a ratio of the amount of the affinity peptide with the collagen binding ability used to the amount of the collagen material used is 1×10⁻⁴-2×10⁻³ μmol: 60 mm³.

Further, the collagen material includes, but is not limited to, a collagen sponge scaffold.

In the present application, the leptin receptor affinity peptide can be ligated to the biological material, thereby improving the recruitment of the biological material to the MSCs, which can play an important role in the field of tissue engineering repair.

Another aspect of the present application is that a leptin receptor affinity peptide obtained by screening in the present application is ligated to a biological material, thereby improving the recruitment of the biological material to MSCs expressing a leptin receptor on a surface and providing an experimental basis for studies on the MSCs in tissue engineering repair and targeted therapy study.

Another aspect of an embodiment of the present application further provides use of the above functional material to preparation of a product with a tissue injury repair function (such as lung injury repair).

Accordingly, another aspect of an embodiment of the present application further provides a product with a tissue injury repair function. The product includes the functional material.

Further, the product has at least a function of being capable of continuously adsorbing MSCs.

By means of the above technical solutions, the leptin receptor affinity peptide provided in the present application has the high affinity for the leptin receptor and can specifically bind to the MSCs. The leptin receptor affinity peptide can be ligated to the biological material so that the affinity polypeptide ligated to the short peptide with the collagen-specific binding ability can specifically bind to the collagen scaffold, thereby improving the recruitment of the biological material to the MSCs expressing the leptin receptor on the surface, obtaining the better tissue injury repair effect and providing the experimental basis for the studies on the MSCs in tissue engineering repair and targeted therapy study.

The technical solutions of the present application are further illustrated in detail below in conjunction with drawings and a number of preferred examples, from which the technical solutions and advantages of the present application are more apparent. Apparently, the described examples are merely part, not all, of examples of the present application. Based on the examples of the present application, all other examples obtained by those of ordinary skill in the art without creative work are within the scope of the present application. Experimental methods without specific conditions noted in the following examples are conducted according to conventional conditions. In addition, if not in conflict, technical features involved in various examples described below of the present application may be combined with each other.

Reagents and raw materials used in the following examples are commercially available, and the experimental methods without the specific conditions noted in the following examples are generally performed according to the conventional conditions or according to conditions recommended by respective manufacturers. In addition, unless otherwise stated, conventional molecular biology, biochemistry, chromatin structure and analysis, analytical chemistry, cell culture and recombinant DNA technology in the technical field and conventional techniques in related fields are used in the experimental methods, detection methods and preparation methods disclosed in the present application.

### Example 1

In this example, a phage display technique is used for screening an affinity polypeptide that specifically binds to a leptin receptor.

A phage random heptapeptide display library is purchased from New England Biolabs (NEB), 100 μL, with a titer of 1× 10¹³ pfu/mL. The phage random heptapeptide display library is stored in a tris-buffered saline (TBS) buffer (50 mM Tris-HCl, 150 mM NaCl [with a pH value of 7.5]) containing 50% glycerol with a library capacity of 1.28×10⁹ transformants. Escherichia coli ER2738 is a host bacterium of this peptide library.

### 1. Coating of Target Protein

A leptin receptor recombinant protein was diluted with a coating buffer (0.1 M sodium bicarbonate buffer [with a pH value of 8.6]) to a final concentration of 100 μg/mL. 100 μL of the mixture was taken and added to a well of an ELISA plate. To ensure that the well plate was wet, the well plate was incubated overnight in a wet box at 4° C.

### 2. Affinity Screening

On the next day, the coating solution in the ELISA plate was poured out, the ELISA plate was patted on clean absorbent paper to completely remove the residual liquid, 200 μL blocking solution (0.1 M NaHCO₃, 5 mg/mL bovine serum albumin (BSA) [with a pH value of 8.6]) was added to each well, and the ELISA plate was placed in the wet box and incubated for at least 1 h at 4° C. The blocking solution was discarded, each well was filled with a TBST buffer (TBS+volume ratio 0.1%[v/v] polysorbate-20 (Tween-20)), the ELISA plate was washed six times with gentle shaking on a decolorizing shaker for 5 min each time, the buffer was poured out, and the ELISA plate was patted dry on clean absorbent paper to remove the residual liquid.

It was required that the experimental step should be performed quickly to avoid drying the well plate in the process. Then, the original library was diluted with a TBST buffer. 100 μL of the diluted library solution was taken and added to the microwell of the ELISA plate pre-coated with the leptin receptor recombinant protein, where the number of added phages was approximately 2×10¹². Subsequently, the ELISA plate was placed on the decolorizing shaker and incubated with gentle shaking for 1-2 h at room temperature, the liquid in the well was discarded, the ELISA plate was patted dry on clean absorbent paper to remove the residual solution, and the plate was washed ten times with a TBST buffer, where the operation was the same as before (to wash away unbound phages). After the last wash, the liquid in the well plate was patted dry on clean absorbent paper, and 100 μL non-specific buffer (0.2 M glycine-hydrochloric acid (glycine-HCl) buffer [with a pH value of 2.2], 1 mg/mL BSA) was added to the microwell of the ELISA plate to elute bound phages. The ELISA plate was placed on the decolorizing shaker and gently shaken for 10 min at room temperature for fully elution. An eluent was collected to a sterile EP tube and neutralized with 15 μL neutralization solution (1 M Tris-HCl [with a pH value of 9.1]). 5 μL of the eluent was taken and used for measuring a titer of eluates according to a conventional M13 titration method, the remaining eluent was added to 20 mL ER2738 bacterium solution which was pre-amplified on the same day and already in an early logarithmic phase, and the first round of amplification was performed on the phage eluates. The mixture was incubated with shaking by the shaker at 220 rpm for 4.5 h at 37° C. The amplified bacterium solution was collected and transferred to a sterile centrifuge tube. After the amplified bacterium solution was centrifuged at 12000 g for 10 min at 4° C., about 80% of phage supernatant was taken and transferred to another sterile centrifuge tube. ⅙ volume of polyethylene glycol (PEG)/NaCl (20%[w/v]PEG-8000, 2.5 M NaCl) was added to the sterile centrifuge tube, which was placed for at least 2 h or overnight at 4° C. to precipitate the phages. After the precipitation was completed, the mixture was centrifuged at 12000 g for 15 min at 4° C., the residual supernatant was discarded with a micropipette, and the mixture can be centrifuged for a short time again to thoroughly suck and discard the supernatant. Precipitates were resuspended in 1 mL TBS buffer. After the mixture was centrifuged again (4° C., 14000 rpm, 5 min), supernatant was collected and transferred to a sterile centrifuge tube. ⅙ volume of PEG/NaCl (20%[w/v]PEG-8000, 2.5 M NaCl) was added to the sterile centrifuge tube, which was incubated on ice for 15-60 min. The mixture was centrifuged at 14000 rpm for 10 min at 4° C., the supernatant was discarded, the mixture was centrifuged again, precipitates were resuspended with 200 μL TBS buffer, and the mixture was microcentrifuged for 1 min to remove insoluble materials, that is, to obtain amplified eluates. 5 μL of the amplified eluates was taken and subjected to the measurement of the phage titer again, a portion of the amplified eluates was left for the second round of affinity screening, and a remaining portion of the amplified eluates was frozen and stored at −20° C. according to a ratio of 1:1 to glycerol for later use. The above steps were repeated for five rounds of screening in total. The numbers of times of washing with TBST in the steps of washing were increased in round-by-round screening.

### 3. Measurement of Phage Titers

ER2738 single colonies were inoculated in 5 mL lysogeny broth (LB) medium, which was incubated on the shaker at 200 rpm for 4.5-5 h at 37° C. to a middle logarithmic phase (OD600 was approximately 0.5). During this period, top agar was heated and dissolved in a microwave oven and distributed into 5 mL sterilized EP tubes in 3 mL/tube. The number of tubes was determined according to the number of phage dilution gradients, and one tube was for each dilution gradient. The distributed top agar was placed in a water bath cauldron at 45° C. for later use. At the same time, LB/IPTG/Xgal culture plates were prepared and placed in an incubator at 37° C. one hour in advance for later use. 10-fold gradient dilution (recommended dilution range: screened eluates without amplification: 10¹-10⁴, amplified phage culture supernatant: 10⁸-10¹¹) was performed on collected phage supernatant by using LB media. After the dilution was completed, the ER2738 bacterium solution in the middle logarithmic phase was distributed into 1.5 mL sterilized EP tubes in 200 μL/tube, where one tube was prepared for each phage dilution. 10 μL of each of the diluted phage solutions with various gradients was taken and immediately added to a respective one of the EP tubes containing the ER2738 bacterium solutions. The mixtures were quickly shaken to be uniformly mixed and incubated for 1-5 min at room temperature. Subsequently, the top agar placed at 45° C. was taken out, and the phage-added ER2738 bacterium solutions were added to the top agar pre-warmed at 45° C. one tube each time. The mixtures were quickly and uniformly mixed and immediately poured on the

LB/IPTG/Xgal culture plates pre-warmed at 37° C. The plates were properly tilted to uniformly distribute the top agar. After cooling and solidification for about 5 min, the plates were inverted and cultured overnight in an incubator at 37° C. Plates with about 100 plaques in total were selected, the numbers of plaques growing out from the plates were counted, and the phage titers (pfu) were calculated. An output/input ratio in each round can be calculated according to the amount of phage input for screening (input titer, Input) in each round and the amount of phage obtained through elution (output titer, Output) in each round so that an enrichment degree of specific phages (recovery efficiency, Recovery) was reflected. After five rounds of screening, it was found that phages that specifically bound to the leptin receptor recombinant protein and had a high affinity were efficiently enriched (see Table 1).

TABLE 1
Input titer, output titer and recovery efficiency in each round

| | Round Number of Screening | | | | |
|---|---|---|---|---|---|
| | 1 | 2 | 3 | 4 | 5 |
| Input | 2 × 10¹² | 2 × 10¹¹ | 1.4 × 10¹¹ | 1.4 × 10¹¹ | 1.4 × 10¹¹ |
| titer | | | | | |
| (pfu) | | | | | |
| Output | 1.5 × 10⁸ | 1.82 × 10⁸ | 6.6 × 10⁷ | 4.8 × 10⁷ | 2.8 × 10⁷ |
| titer | | | | | |
| (pfu) | | | | | |
| Recovery | 7.5 × 10⁻⁵ | 9.1 × 10⁻⁵ | 4.7 × 10⁻⁴ | 3.4 × 10⁻⁴ | 2 × 10⁻⁴ |
| efficiency | | | | | |

### 4. Preparation and Sequencing of Phage Monoclonal DNA

When the screening was performed until the third, fourth and fifth rounds, after titration was performed on phage amplification obtained in each round, 15 phage monoclones were randomly selected in each of the titer plates, and rapid purification of a sequencing module was performed on the phage monoclones after amplification to produce a sufficiently pure template to be sent to a biological company for gene sequencing: an ER2738 amplified bacterium solution cultured overnight (an OD value was about 0.5) was inoculated in an LB medium according to 1:100, shaken to a logarithmic phase and distributed into culture tubes in 1 mL/tube. A phage monoclonal blue plaque was randomly selected and added to the above 1 mL culture tube. 15 monoclones in total were selected for amplification in each round. After the mixture was cultured on the shaker at 250 rpm for 4.5-5 h at 37° C., the incubated phage bacterium solution was transferred to a microcentrifuge tube and centrifuged at 10000 rpm for 30 s. Supernatant was transferred to a new centrifuge tube and centrifuged again. 80% of the supernatant was sucked and transferred to a new centrifuge tube, and the supernatant was an amplified phage stock solution. If subsequent experiments were not performed immediately, the amplified phage stock solution can be temporarily stored at 4° C. If the amplified phage stock solution needed to be stored for a long time, the amplified phage stock solution should be diluted with glycerol at 1:1 and stored at −20° C.

Rapid purification of sequencing template: after the phage monoclonal amplification was performed according to the above method, the above collected supernatant containing the phages (500 μL) was taken, 200 μL PEG/NaCl was added to the above collected supernatant, the mixture was turned upside down to be uniformly mixed, placed for 10-20 min at room temperature and centrifuged at 14000 rpm for 10 min at 4° C., the supernatant was discarded, and the centrifuge operation can be performed again to thoroughly suck and discard the residual supernatant. Precipitates were thoroughly resuspended in 100 μL iodide buffer (10 mM Tris-HCl, 1 mM ethylenediaminetetraacetic acid (EDTA), 4 M Nal [with a pH value of 8.0]), 250 μL ethanol was added to the mixture, and the mixture was incubated for 10-20 min at room temperature (to precipitate DNA). The mixture was centrifuged at 14000 rpm for 10 min at 4° C., and supernatant was discarded. The precipitates were washed once with 50 μL precooled 70% ethanol, and the mixture was centrifuged again. After supernatant was discarded, a cap was removed for air drying so that the ethanol was fully volatilized. Finally, the precipitates were resuspended in 30 μL double-distilled water, and the mixture was the sequencing template. 5 μL of the sequencing template was taken and detected through 1% agarose gel electrophoresis. Qualified DNA after the detection was sent to the company for the sequencing with a selected—96gIII sequencing primer.

Through comparison, the inventors of this case obtained a short peptide with the highest frequency of occurrence among the monoclones which were randomly selected and sent for the sequencing in several rounds of screening. The short peptide, which was named “HY7 peptide”, had an amino acid sequence of SEQ ID No. 1: HGGVRLY.

### 5. Detection of Binding Ability of HY7 Phage Monoclone to Target Molecule

The HY7 phage monoclone obtained in this example was amplified according to the phage amplification method. At the same time, a control M13KE negative control phage (purchased from NEB, 40 μL, 1×10¹³ pfu/mL) was amplified. The amplified HY7 phage monoclone and M13KE negative control phage were titrated according to the above titration method to obtain amplified phage concentrations. The target protein, that is, the leptin receptor recombinant protein, was diluted with a coating buffer to a final concentration of 100 μg/mL one night in advance. 100 μL of the mixture was taken and added to a well of an ELISA plate. To ensure that the well plate was wet, the well plate was incubated overnight in the wet box at 4° C. The HY7 phage monoclone and the M13KE negative control phage were added to the well containing the target protein for binding and elution according to the step of screening the phages to obtain an eluent of the HY7 phage binding to the target protein and an eluent of the M13KE negative control phage binding to the target protein, respectively. The eluent of the HY7 phage binding to the target protein and the eluent of the M13KE negative control phage binding to the target protein were titrated according to the phage titration method. The binding of the HY7 phage monoclone to the target molecule and the binding of the M13KE negative control phage to the target molecule were compared. It can be seen that compared with the M13KE negative control phage, the HY7 phage monoclone has a larger binding amount to the leptin receptor recombinant protein (as shown in FIG. 1), where the ordinates show binding recovery efficiency of the HY7 phage monoclone and the M13KE negative control phage to the leptin receptor recombinant protein, that is, ratios of the amounts of phage recovered through the elution after the binding to the amounts of input phage.

### Example 2 HY7 can Specifically Bind to Human MSCs

### 1. Detection of Binding Ability of HY7 Phage Monoclone to Human MSCs

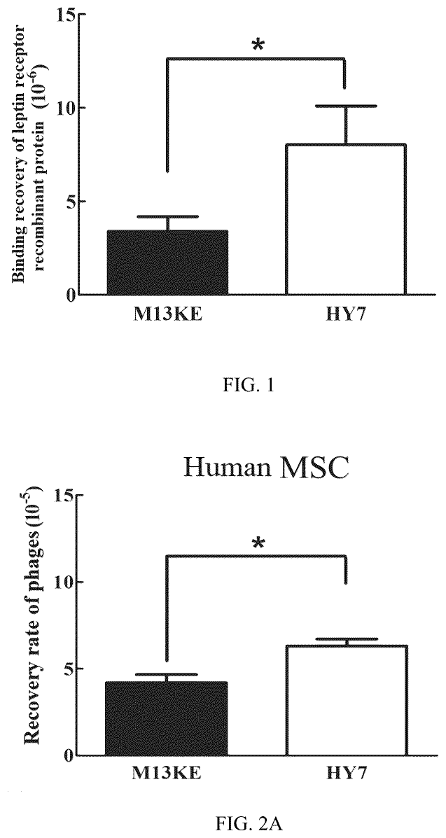

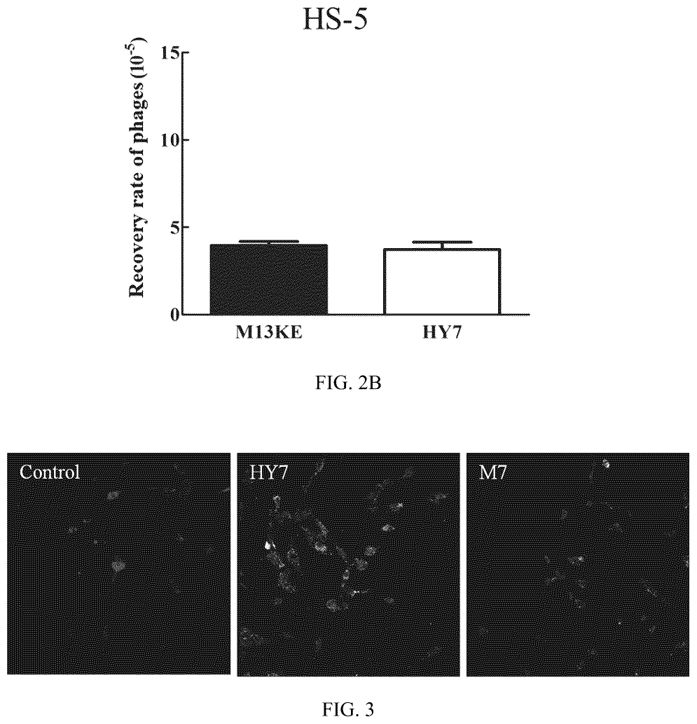

An HY7 phage monoclone and an M13KE negative control phage were amplified with reference to the method in Example 1 and titrated. The HY7 phage monoclone and the M13KE negative control phage were added to well plates containing human MSCs with a confluence pre-cultured to about 80%, and an unrelated human bone marrow stromal cell line HS-5 was set as a control. The HY7 phage monoclone and the M13KE negative control phage were subjected to binding and elution according to a step of screening phages to obtain an eluent of the HY7 phage binding to the target protein and an eluent of the M13KE negative control phage binding to the target protein, respectively. The eluent of the HY7 phage binding to the target protein and the eluent of the M13KE negative control phage binding to the target protein were titrated according to a phage titration method. The binding of the HY7 phage monoclone to the target molecule and the binding of the M13KE negative control phage to the target molecule were compared. It can be seen from the results that HY7 can specifically binds to surfaces of the MSCs but does not bind to the control cells HS-5. FIG. 2A is a schematic diagram illustrating the binding recovery of an HY7 phage monoclone and an M13KE negative control phage to human MSCs, and FIG. 2B is a schematic diagram illustrating the binding recovery of an HY7 phage monoclone and an M13KE negative control phage to HS-5 cells.

### 2. Binding of HY7 to Human MSCs Detected by Fluorescence Microscope

A company was entrusted to synthesize a fluorescein isothiocyanate (FITC)-labeled polypeptide (FITC-HY7) in vitro, and a heptapeptide reported in the literature that did not bind to MSCs was selected to synthesize an FITC-labeled negative reference polypeptide (FITC-M7) at the same time. Human MSCs and HS-5 cells were separately digested and seeded in 6-well plates at a density of 5×10⁵ cells/well, respectively. After the cells were cultured in an incubator for 24 h at 37° C., culture solutions were discarded, and the well plates were washed three times with (PBS). After the cells were pre-labeled with a DiI dye for 15 min, the well plates were washed three times with PBS, media were replaced with serum-free media, and the above synthesized FITC-HY7 peptide (5 μmol/L) and FITC-M7 peptide (5 μmol/L) were added to the well plates containing the cells, respectively. At the same time, the cells not in peptide were used as a blank control. The cells were co-incubated for 30 min at 37° C., and the well plates were washed three times with PBS. After the cells were fixed with 4% paraformaldehyde for 20 min, the well plates were washed three times with PBS, DAPI (1:10000 dilution) was added to the well plates, and the cells were incubated for 10 min. After the plates were washed three times with PBS, the binding of the fluorescent polypeptide cells FITC-HY7 and FITC-M7 to the human MSCs was observed under a confocal fluorescence microscope using the polypeptide-unadded group as a control. The results are shown in FIG. 3, where a binding amount of the FITC-HY7 peptide to the human MSCs is significantly larger than that of the negative control peptide.

### 3. Binding of HY7 to Rat MSCs Detected by Flow Cytometry

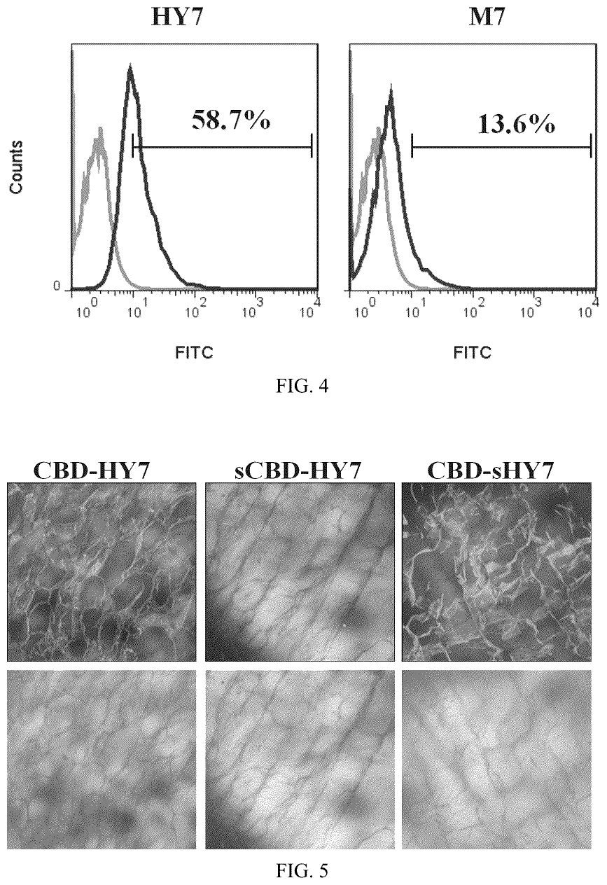

To evaluate the species specificity of the HY7 peptide and provide a basis for subsequent animal experiments, the binding of HY7 to rat MSCs was specifically verified. Rat bone marrow cells were acquired according to a conventional method and subjected to culture and identification, and rat MSCs were obtained. The binding of an FITC-HY7 peptide and an FITC-M7 peptide to the rat MSCs was detected according to the above flow cytometry detection method. The results are shown in FIG. 4, where a binding ability of HY7 to the rat MSCs is significantly stronger than that of M7.

### Example 3 CBD-Affinity Peptide can Bind to Collagen Material for Lung Injury Repair

### 1. CBD-Affinity Peptide can Bind to Collagen Material

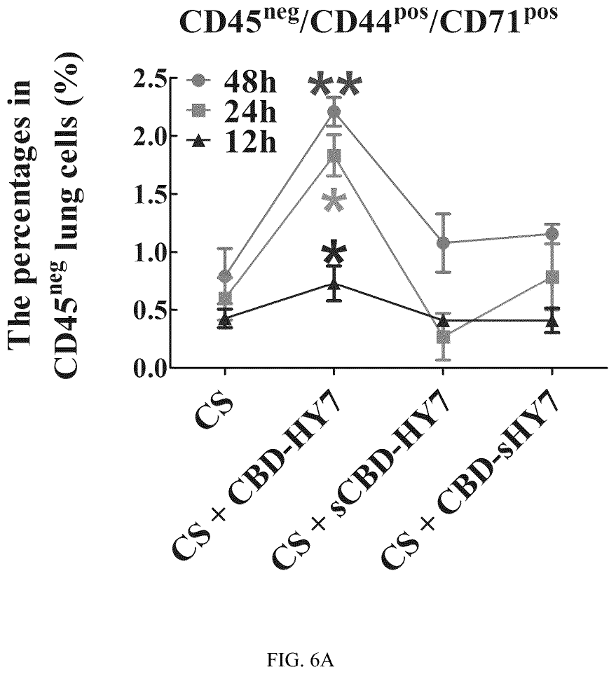

To avoid that an affinity peptide cannot effectively stay on a biological material to maintain an effective concentration due to a simple adsorption method and the affinity peptide was affected and did not perform an effect, in the present application, a short peptide CBD with a collagen-specific binding ability (a sequence was specifically SEQ ID No. 3: TKKTLRT) was introduced while the affinity polypeptide was synthesized. The short peptide CBD was synthesized on an N-terminus of the affinity polypeptide and ligated to the affinity polypeptide through a linker whose sequence was specifically SEQ ID No. 5: GGG-K-FITC. To evaluate whether the CBD-affinity peptide can effectively bind to the collagen material, FITC-labeled CBD-HY7 was synthesized at the same time, and for ease of control, an FITC-labeled CBD misalignment control (sCBD-HY7) and HY7 misalignment control (CBD-sHY7) were also separately synthesized. Specific experimental method: collagen sponge sheets with a diameter of 5 mm were fully wetted with PBS, the excess liquids were absorbed with sterile filter paper, and dissolved FITC-labeled affinity peptide and control peptides were uniformly added dropwise to the corresponding collagen sponge sheets, respectively (with a concentration of 5-100 μmol/L), where a ratio of the amount of FITC-labeled affinity peptide used to the amount of collagen sponge sheet used was 1×10⁻⁴-2×10⁻³ μmol: 60 mm³, which ensured the fully absorption of the collagen sponges and no spilling of the liquids. The collagen sponges were incubated for 2 h at 37° C. and washed three to five times with PBS. The binding of CBD-HY7, sCBD-HY7 and CBD-sHY7 to the collagen sponges was shot by a confocal fluorescence microscope. The results are shown in FIG. 5, where the peptides expressing the CBD can specifically bind to the collagen sponges while the misalignment CBD control loses an ability to bind to the collagen sponge.

2. Implantation of Collagen Sponge Bound with CBD-Affinity Peptide into Rat Lung Injury Site can Recruit Endogenous MSCs in a Short Time

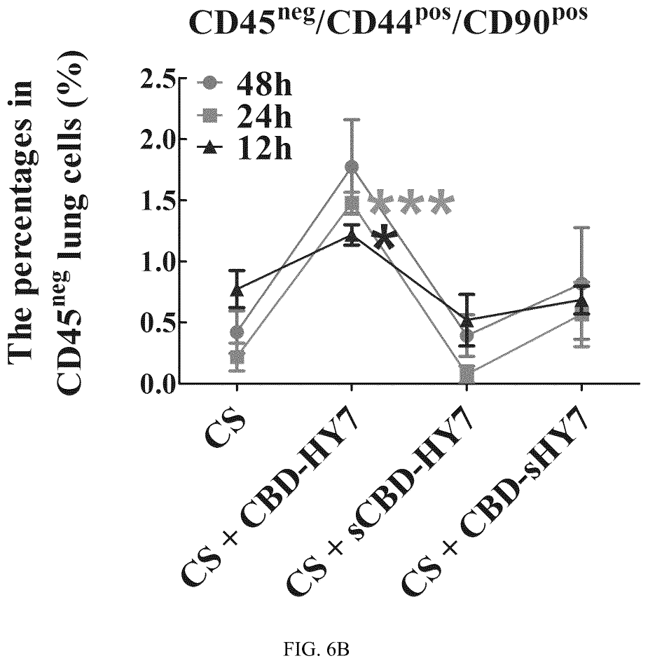

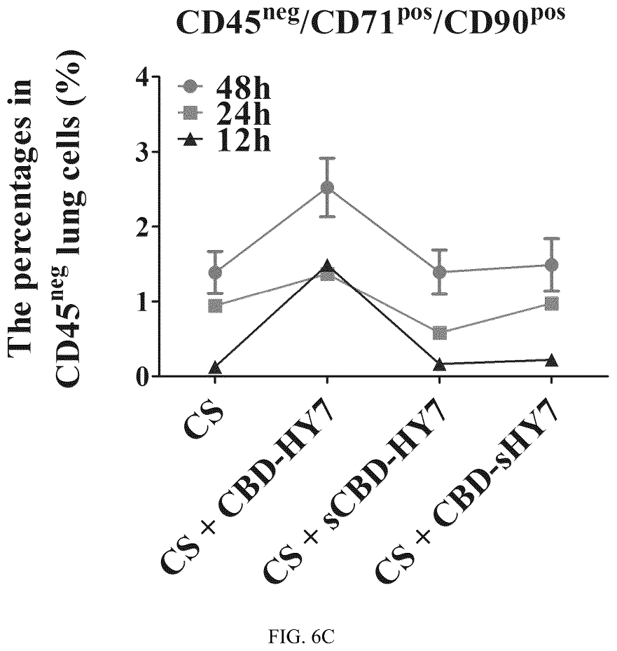

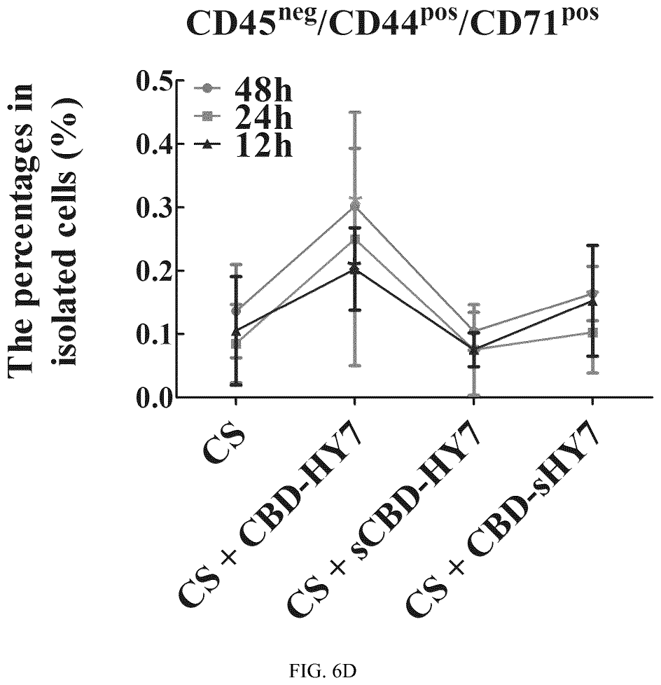

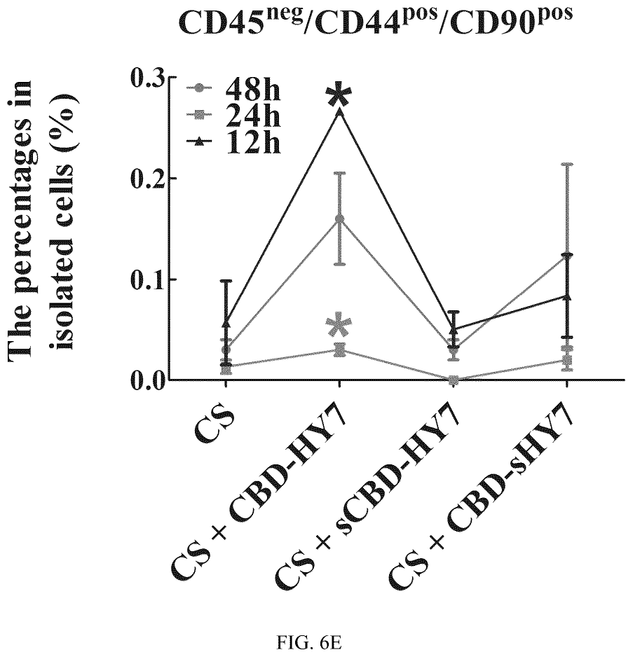

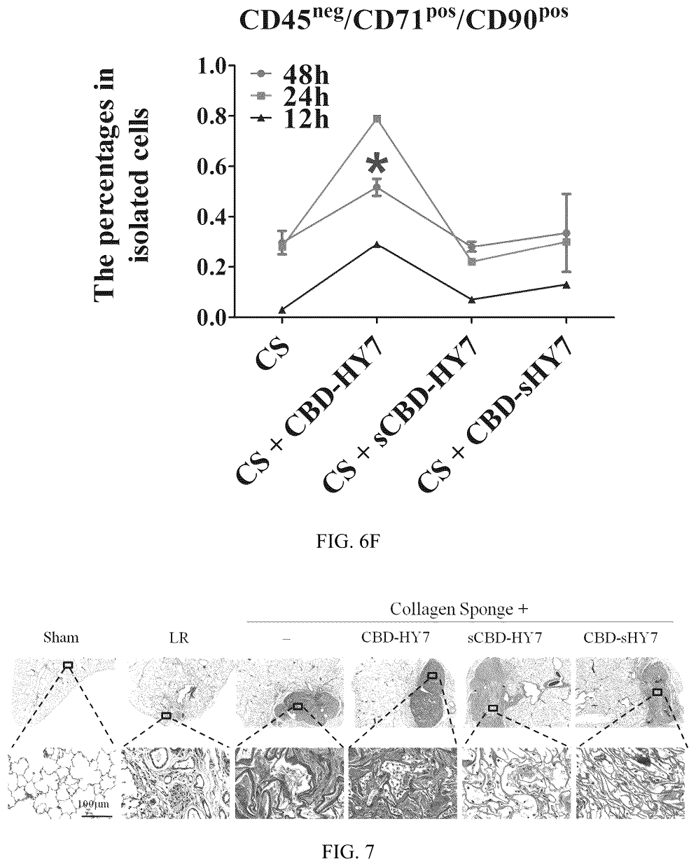

Rats weighing about 150 g were anesthetized, intubated and subjected to partial resection on middle lobes of right lungs in the case of positive pressure ventilation of ventilators, where lung tissues with a size of about 0.5 cm*0.5 cm*0.3 cm were resected, and the collagen sponges which had been uniformly added dropwise with the CBD-affinity peptide or the control peptides and incubated for 2 h at 37° C. in advance were implanted into rat lung defect sites, respectively. After the rats were sacrificed after 12 h, 24 h or 48 h, respectively, the implanted collagen sponges were completely separated and taken out. The collagen sponges were dissolved with a collagenase, and cells adsorbed on the collagen sponges in vivo were separated and collected. Through identification of expression of CD45, CD44, CD71 and CD90 on surfaces of the cells, proportions of the numbers of MSCs adsorbed on the collagen sponges at the implantation sites were analyzed. The proportions that the MSCs accounted for in the cells digested from the collagen sponges were detected by flow cytometry. The results are shown in FIGS. 6A to 6F, where CBD-HY7 can adsorb more MSCs to the collagen sponge in a short time compared with the control groups.

3. Implantation of Collagen Sponge Bound with CBD-Affinity Peptide into Rat Lung Injury Site can Repair Lung Injury

After a CBD-affinity peptide and control peptides were implanted into rat lung injury sites according to the above method, lung tissues were taken out 35 days later and subjected to pathological identification. It can be found that compared with the control groups, the tissue site where the CBD-affinity peptide was implanted has a better repair effect. The results of Masson's staining of the lung tissues and the collagen sponge regions are shown in FIG. 7.

In summary, in the present application, a leptin receptor-specific binding polypeptide is obtained through the phage display technique, and it is proved that the polypeptide can be targeted to the human MSCs with a high affinity. Moreover, in a process of identifying the species specificity of the affinity polypeptide, it is found that the polypeptide has no apparent species specificity. Therefore, it is assumed that the affinity polypeptide has a wide application range. Then, the CBD is synthesized to the leptin receptor affinity peptide obtained through the screening, and the affinity peptide can target and bind to the collagen material through the CBD, so that the MSCs can be specifically adsorbed and recruited, and when the collagen material is implanted in vivo as a scaffold for the lung injury repair, the MSCs can be continuously adsorbed, thereby obtaining a better repair effect.

Although the present application has been described with reference to illustrative examples, it will be understood by those skilled in the art that various other changes, omissions and/or additions may be made and elements of the examples may be replaced with substantial equivalents without departing from the spirit and scope of the present application. In addition, many modifications may be made without departing from the scope of the present application so that particular situations or materials adapt to teachings of the present application. Therefore, it is not intended herein that the present application be limited to the examples disclosed for performing the present application, but it is intended that the present application will include all examples belonging to the scope of the appended claims.

The ASCII text file “Sequence.txt” created on Dec. 18, 2023, having the size of 1099 bytes, is incorporated by reference into the specification.

1. An affinity peptide with a collagen binding ability, comprising: a leptin receptor affinity peptide and a short peptide with a collagen-specific binding ability; wherein the short peptide with the collagen-specific binding ability is ligated and bound to an N-terminus of the leptin receptor affinity peptide through a linker, a sequence of the leptin receptor affinity peptide is as shown in SEQ ID No. 1, the short peptide with the collagen-specific binding ability is a collagen binding domain (CBD) whose sequence is as shown in SEQ ID No. 3, and a sequence of the linker is as shown in SEQ ID No. 4 or SEQ ID No. 5.

2. The affinity peptide with the collagen binding ability according to claim 1, wherein the leptin receptor affinity peptide is obtained by using a phage display technique with a leptin receptor recombinant protein for affinity screening followed by screening in a random heptapeptide library.

3. A functional material, comprising a collagen material and the affinity peptide with the collagen binding ability according to claim 1, wherein the affinity peptide with the collagen binding ability binds to a surface and/or an interior of the collagen material.

4. The functional material according to claim 3, wherein a ratio of an amount of the affinity peptide with the collagen binding ability used to an amount of the collagen material used is 1×10⁻⁴-2×10⁻³ μmol: 60 mm³.

5. The functional material according to claim 3, wherein the collagen material comprises a collagen sponge scaffold.

6. Use of the functional material according to claim 3 to preparation of a product with a tissue injury repair function.

7. A product with a tissue injury repair function, comprising the functional material according to claim 3.

8. The product according to claim 7, wherein the product has at least a function of being capable of continuously adsorbing mesenchymal stem cells.
